(19) United States
(12) Patent Application Publication
(10) Pub. No.: US 2007/0068651 A1
(43) Pub. Date: Mar. 29, 2007

(54) Laminated foam temperature regulation device

(75) Inventors: Clifford E. Gammons, Loudon, TN (US); Scott Gammons, Loudon, TN (US); Michael V. Sassoon, Pardes Hanna-Karkur (IL); Alexander Barash, Tzoran (IL); Michael Rumack, Pardes Hanna-Karkur (IL)

Correspondence Address: PITTS AND BRITTIAN P C, P O BOX 51295, KNOXVILLE, TN, 37950-1295, US

(73) Assignees: Adroit Medical Systems, Inc., Loudon, TN (US); MTRE Advanced Technologies, Ltd., Or-Akiva (IL)

(21) Appl. No.: 11/235,432

(22) Filed: Sep. 26, 2005

(51) Int. Cl. A61F 7/00

(52) U.S. Cl. 165/46; 607/108

(57) ABSTRACT

A heat exchange apparatus for providing temperature regulation to a person, animal, or inanimate object. The heat exchange device includes an inlet receiving a temperature controlled fluid that is distributed to and passes through a porous layer before being collected and discharged through an outlet. The porous layer is encapsulated in a pair of non-porous layers or sheets. In one embodiment, the pair of non-porous layers extend beyond the boundaries of the porous layer and form a pair of manifolds for distributing the inlet and outlet fluid to and from the porous layer. In one embodiment, the manifolds include flow diverters for distributing the fluid to and from the porous layer. In various embodiments, the heat exchange apparatus is adapted for wrapping around a body part or object, for securing to a seat, and for wearing as a vest or other article of clothing.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention pertains to a heat exchange device for regulating temperature. More particularly, this invention pertains to a readily deformable heat exchange device and system applied on a person, an animal, or attached to an inanimate object.

2. Description of the Related Art

During lengthy surgical procedures, an externally applied heat exchange device is utilized to heat or cool selected portions of a patient's body during an operation lasting greater than approximately an hour in order to regulate the patient's core temperature. Heat exchange devices are also utilized for reclining patients during rehabilitation from surgery, or when bed-ridden due to a chronic illness, with heating or cooling treatment applied by one or more heat exchange devices positioned on, under, or encircling a patient's extremity to control the patient's core temperature. A typical device includes a heat exchange blanket sized to fit under a patient's torso when reclining, or a plurality of heat exchange pads wrapped in encircling relationship around selected portions of the patient's body. The prior art typically discloses a blanket or pad providing fluid transport within channels defined by a plurality of tubular channels interior of, or on an exterior surface of the device, and having one-way entry and outlet ports for attaching tubing extending to a pumping device and a fluid heating or cooling device.

Prior heat exchange pad devices typically have a pair of outer plastic and/or fabric layers sandwiched together with a serpentine tubular passageway positioned therebetween to allow passage of heating or cooling fluids in one direction from an entry port to an outlet port of the heat exchange pad. The surface of these devices are typically composed of an array of insulating, thermally inactive islands surrounded by circuitous fluid passages that exert pressure on the skin. The percentage of thermally inactive surface typically ranges between 20 to 50%. It has been observed that these devices with thermally inactive islands, when applied against a patient's body for periods of time greater than about one hour, will create uneven heating and pressure patterns on the patient's skin leading to indentations and “hot spots” in the patient's skin. To prevent uneven heating patterns on a patient's skin during extended medical procedures, or on a comatose patient, prior heat exchange pads required turning a patient relative to the heat exchange pad, and/or adjusting the position of the pad to physically change the heating patterns across the patient's skin. Additional difficulties with prior pads typically include creation of skin indentations by hard outer areas of a pad formed by pressure dilation of the interior tubing during passage of fluids therein. Additional complications with prior devices include non-planar and/or partially rigid pad surfaces formed by uneven distribution of interior tubing in the pad, resulting in outer surfaces not conforming to natural curvatures of the patient's extremity, front or back torso, and/or sides of the patient's torso.

Prior heat exchange devices have included “hot spots” near an inlet portal, and “cool zones” next to an outlet portal when heated fluids flow through the device due to the inlet flow channels being inadequately sized to rapidly disburse heating fluids within and across a full width or length of the interior, therefore leading to overheating of the body proximal to the entry port, and inadequate heating of the body along the outlet port. The prior art devices with circuitous fluid passages are prone to blockage due to misuse such as folding, crimping, or excess weight on a small area of the device. Full or partial blockage results in elevated fluid pressure in the device, compromised flow to certain parts of the device, reduced effective heat transfer area and premature device failure. To prevent blockage, it is necessary to check the device periodically, move the patient when a blockage is observed and carefully monitor the fluid pressure in the device.

An improved thermal treatment device is sought to provide for heating or cooling of animate objects or inanimate devices requiring thermal regulation. An improved thermal treatment device is sought to provide for induced hyperthermia or hypothermia for humans or animals. In addition, an improved thermal treatment device is sought to provide thermal regulation for, and pressure management against, a human or animal requiring heating or cooling without allowing overheating or underheating of the skin over extended use and without the risk of blockage, compromised flow, and elevated fluid pressure.

An improved thermal treatment device is provided for efficient heat transfer to/from a human, an animal, or an inanimate device. The thermal treatment device reduces resistance to heat transfer by providing a thin, readily pliable and conformable, and thermally active contacting surface for disposition against the object of interest. Simultaneously, pressure management is provided to reduce localized pressure on body parts such as bony prominences for extended time periods. An improved system is also provided for efficient heating or cooling to control a person's core temperature and ensuring body thermoregulation during medical treatment or extensive activities in heated or cooled environments, without producing localized pressure and/or heat induced indentations or bruising on the skin and without inducing variable temperatures across the covered skin.

### BRIEF SUMMARY OF THE INVENTION

According to one embodiment of the present invention, a heat exchange device for providing temperature regulation for a person's body, an animal's body, and/or an inanimate object is provided. The heat exchange device is a pliable pad with an inlet receiving a fluid at a specified temperature, a porous layer sandwiched between two non-porous layers, and an outlet for discharging the fluid after it flows through the porous layer. The porous layer is an open-cell foam through which a fluid flows.

In one embodiment, the inlet connects to an inlet manifold that distributes the fluid across an edge of the porous layer. The outlet likewise connects to an outlet manifold that collects the fluid passing out of the porous layer. The manifolds ensure the distribution of fluid across the full width of the porous layer; thereby ensuring even heat exchange across a large area of the heat exchange device.

In various embodiments, the heat exchange device is adapted for use as a pad with one or more inlets and outlets located at various positions on the pad, as a pad adapted to be wrapped around an extremity or object to be temperature regulated, as a pad adapted to be used in conjunction with a seat, and as a vest or other garment adapted to be worn.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view of one embodiment of the heat exchange device;

FIG. 2 is a plan view of the embodiment illustrated in FIG. 1;

FIG. 3A is a cross-section view of one embodiment of the heat exchange device;

FIG. 3B is an exploded side view of one embodiment of the porous layer;

FIG. 3C is an exploded side view of one end of one embodiment of the heat exchange device;

FIG. 4 is a plan view of another embodiment of the heat exchange device with an adjacent inlet and outlet;

FIG. 5 is a plan view of another embodiment of the heat exchange device covering a person;

FIG. 6A is a side view of still another embodiment of the heat exchange device;

FIG. 6B is a cross-section view of the embodiment of FIG. 8;

FIG. 7 is a perspective view of another embodiment of the heat exchange device adapted to fit on a seat;

FIG. 8 is a front view of still another embodiment of the heat exchange device adapted to be worn as an article of clothing FIG. 9 is a plan view of another embodiment of a heat exchange device; and

FIG. 10 is an exploded side view of the embodiment illustrated in FIG. 9.

### DETAILED DESCRIPTION OF THE INVENTION

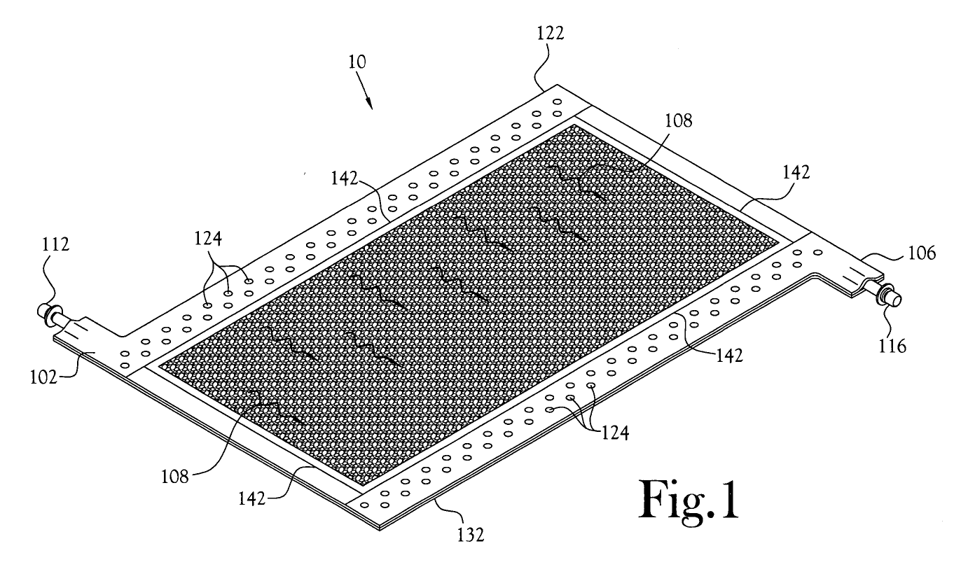

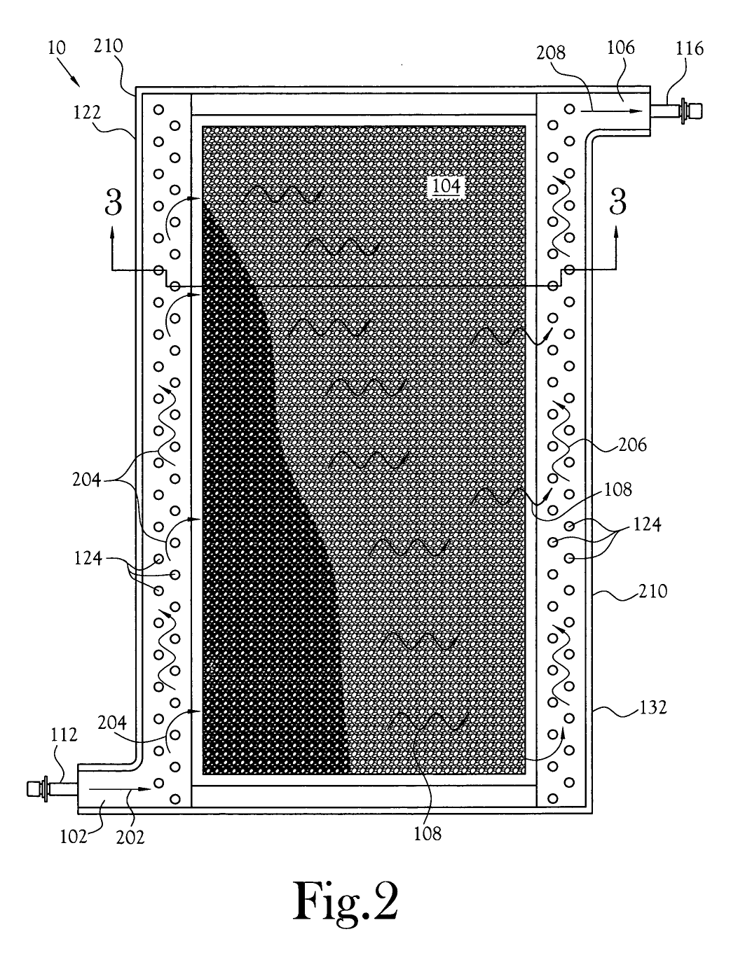

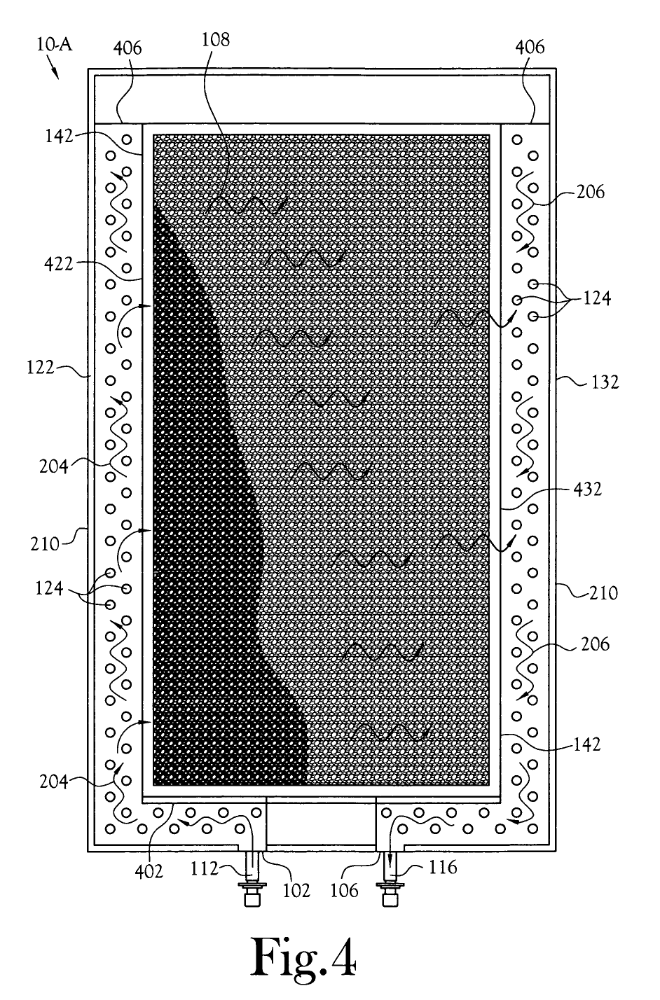

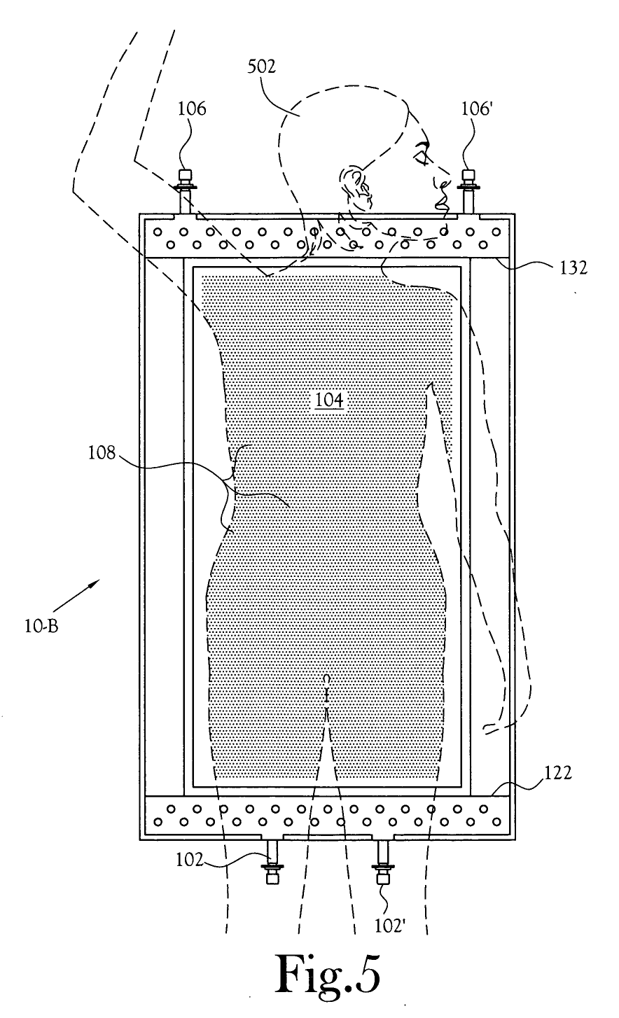

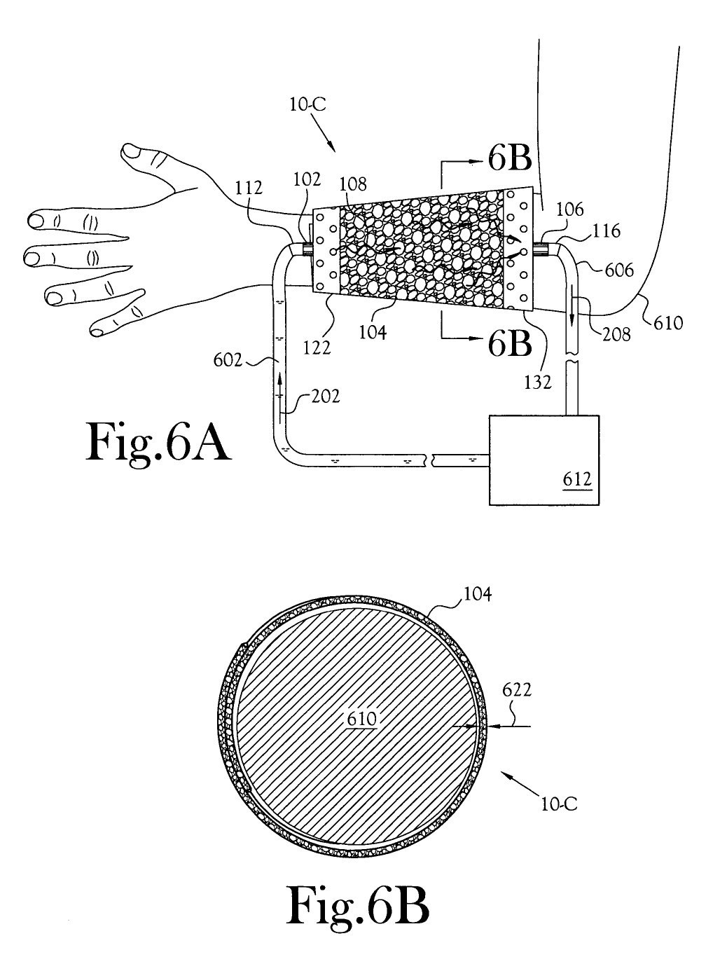

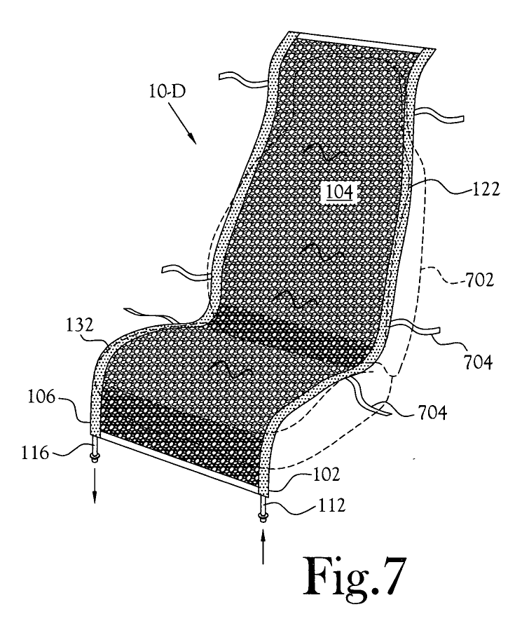

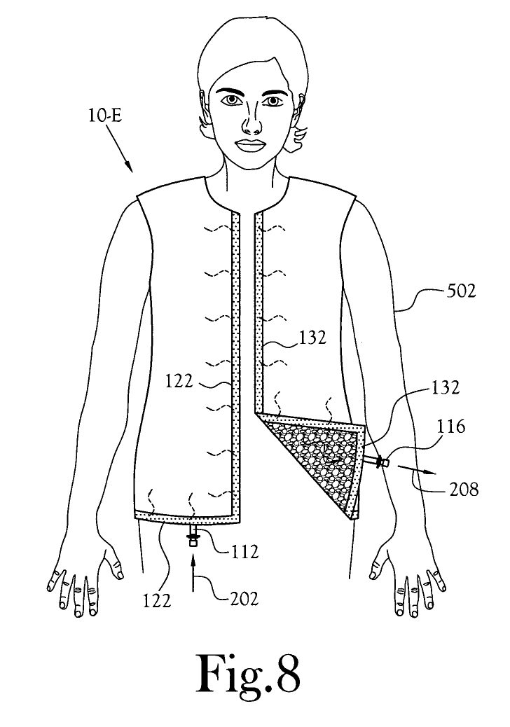

An apparatus for providing temperature regulation for a person's body, an animal's body, and/or an inanimate object is disclosed. The heat exchange device is referred herein generally as 10. Those skilled in the art will recognize that, although the description contained herein may identify the use of an embodiment of the heat exchange device 10 with respect to a patient, a human, an animal, or an inanimate object, the device is not limited to be used only with the identified subject, but is suitable for use with any object, including a person, an animal, and an inanimate object. The device 10 is illustrated in various embodiments for a variety of uses, including use as a readily conformable temperature regulation pad, or heat exchange device, 10 illustrated in FIGS. 1 and 2, as a planar blanket 10-A, 10-B illustrated in FIGS. 4 and 5, as an encircling device 10-C illustrated in FIGS. 6A and 6B, as a seat cover 10-D illustrated in FIG. 7, and as a vest 10-E illustrated in FIG. 8. However, the heat exchange device 10 is not limited to only the uses and applications illustrated.

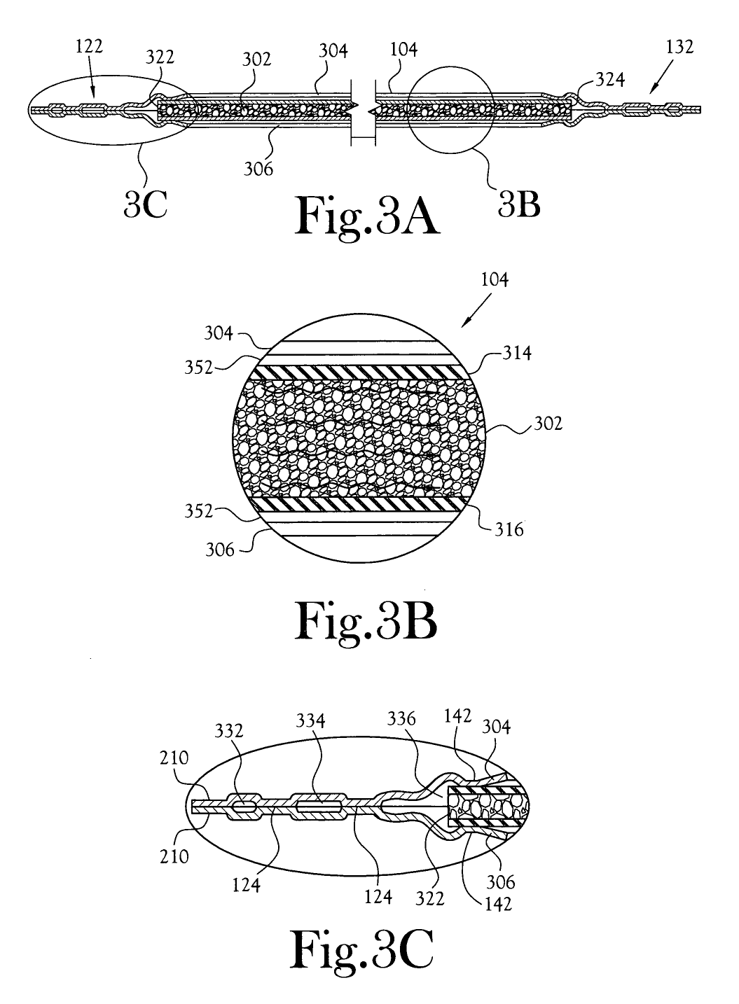

The heat exchange device 10 is a generally planar, flexible pad that conforms to the shape of the object to be temperature controlled. FIG. 1 illustrates a perspective view of one embodiment of the heat exchange device 10. FIG. 2 illustrates a plan view of the embodiment illustrated in FIG. 1. FIG. 3A illustrates a cross-section view of one embodiment of the heat exchange device 10. FIG. 3B illustrates an exploded side view of one embodiment of the porous layer 302. FIG. 3C illustrates an exploded side view of one end of one embodiment of the heat exchange device 10.

As seen in FIGS. 1 and 2, the heat exchange device, or pad, 10 includes an inlet 102 for receiving a temperature controlled fluid, a heat exchange portion 104 through which the fluid 108 flows, and an outlet 106 for discharging the fluid exiting the heat exchange portion 104. In the illustrated embodiment, the inlet 102 is located diametrically opposite the outlet 106. Those skilled in the art will recognize that the location of the inlet 102 and the outlet 106 can vary without departing from the spirit and scope of the present invention.

Referring to FIGS. 3A and 3B, the heat exchange portion 104, in the illustrated embodiment, is constructed of a porous layer, or sheet, 302 sandwiched between two non-porous layers, or sheets, 314, 316. The porous layer 302 has an upper and lower surface in which the outer and inner non-porous layers 314, 316 are adjacent. In one embodiment, the non-porous sheets 314, 316 are attached to the porous layer 302. In this embodiment, the non-porous sheets 314, 316 are attached to the upper and lower surface of the porous layer 302 by adhesive, flame bonding, heat sealing, or other methods of attaching one sheet to another that prevents the non-porous sheets 314, 316 from separating from the porous layer 302, regardless of pad 10 deformation during positioning to conform to curvatures of the person, animal, or object requiring thermal regulation. In another embodiment, the porous layer 302 has a skin 314, 316 on the two opposing surfaces, and the skin 314, 316 is non-porous and impermeable to the fluid 108 25 flowing through the porous layer 302.

In one embodiment, the porous layer 302 is an open-cell foam, for example, a flexible cellular polyurethane or a similar synthetic hydrophilic polymer.

The porous layer 302 has a foam structure in which the cells of the foam are interconnected. The connections between the cells allow for tortuous flow of fluid 108 through the porous layer 302. In various embodiments, the porous layer 302 includes an open cell flexible polyurethane foam material with a density less than 2.0 lb/cu. ft., with less than 120 pores per inch, an elongation greater than 300%, either reticulate or non-reticulate structure, either standard or hydrophilic composition, and having a plurality of internal fluid channels extending between a matrix of interconnecting cell units. The plurality of fluid channels are randomly oriented to provide generally aligned and directed fluid flow paths across the porous layer 302 length and width between the inlet manifold 122 end and the outlet manifold 132 end. As fluid 108 enters the porous layer 302, the cells fill with fluid 108 due to both a wicking effect and a pressure differential across the cells. Upon filling the cells expand and reduce resistance to flow. In various embodiments, the porous layer 302 structure experiences inflation from 25% to 100% of its original, unfilled thickness, depending upon the internal pressure of the fluid 108. Several factors contribute to the integrity of the heat exchange portion 104, including the adhesion of the polyurethane foam of the porous layer 302 to the impervious non-porous layers 314, 316, the elongation of the foam of the porous layer 302, and the flexibility of the non-porous layers 314, 316.

The two non-porous layers 314, 316 are pliable sheets that are impervious and impermeable to the fluid 108. In various embodiments, the two non-porous layers 314, 316 are flexible polyurethane or a synthetic polymer material. In another embodiment, the outer non-porous layer 314, that is, the layer 314 that is not positioned adjacent the object to be temperature controlled, is a pliable material that has limited stretch and the inner non-porous layer 316, that is, the layer 316 that is positioned adjacent the object to be temperature controlled, is a pliable material that is stretchable or elastic. In this embodiment, the inner non-porous layer 316 is forced against the object to be temperature 25 controlled by the expansion of the porous layer 302 by the fluid 108 flowing through the layer 302 because the outer non-porous layer 314 does not expand to accommodate the expansion of the porous layer 302.

In one embodiment, the porous layer 302 has an initial thickness of between about 0.08 inches and about 0.24 inches when fluid 108 is not flowing and the porous layer 302 is not pressurized by the application of a fluid 202 to the inlet 102. When a pressure differential is applied across the inlet 102 and the outlet 106, the fluid 202 flows from the inlet 102, through the manifold 122, and into the porous layer 302.

In one embodiment, a water flow rate of 0.25 to 0.50 gallons per minute results in a typical range of internal pressure maintained by the porous layer 302 of between two and five pounds per square inch. The pressure of the fluid 108 in the porous layer 302 causes the porous layer 302 to expand in thickness. In various embodiments, the thickness of the porous layer 302 increases by 0.02 to 0.24 inches. This increase in thickness causes a displacement of the non-porous layers 314, 316 normal to the plane of the porous layer 302. In the embodiment in which the heat exchange device 10 is wrapped around or secured to a patient, for example, the encircling device 10-C illustrated in FIGS. 6A and 6B, the increase in thickness causes a compressive effect on the patient. That is, as the porous layer 302 expands to increase its thickness, the expansion forces the inner non-porous layer 316, 314 against the patient.

Referring to FIG. 3A, the illustrated embodiment shows the heat exchange portion 104 with a first end, or side, 322 mated with an inlet manifold 122 and a second end, or side, 324 mated with an outlet manifold 132. The fluid 108 travels from the first end 322 to the second end 324 of the heat exchange portion 104. The inlet manifold 122 is positioned between the inlet 102 and the heat exchange portion 104, and the outlet manifold 132 is positioned between the heat exchange portion 104 and the outlet 106. In the embodiment shown in FIG. 2, each manifold 122, 132 extends fully along one of two opposite sides of the heat exchange portion 104. The manifolds 122, 132 include flow diverters 124 that distribute flow of the fluid 204, 206 in the manifolds 122, 132. The fluid 204 flows around and past the flow diverters 124 as the fluid 204 flows from the inlet 102 into the porous layer 302. The fluid 108 flows through the porous layer 302 into the outlet manifold 132, where the fluid 206 flows around and past the flow diverters 124. The fluid 208 flows out the outlet 106 after leaving the manifold 132.

The inlet manifold 122 and the outlet manifold 132 are formed of two manifold sheets 304, 306 of a pliable non-porous material that is impermeable to the fluid 202, 204, 108, 206, 208. In the illustrated embodiment, the manifold sheets 304, 306 are large sheets that extend past the edge of the heat exchange portion 104 and have their central portion cut out. The manifold sheets 304, 306 are sealed near the perimeter of the non-porous sheets, or layers, 314, 316 of the heat exchange portion 104. In other embodiments, the manifold sheets 304, 306 are each sheets without their central portion cutout, and the manifold sheets 304, 306 are bonded to the surface of the non-porous sheets, or layers, 314, 316 of the heat exchange portion 104 or the portion of the sheets 304, 306 adjacent the perimeter of the heat exchange portion 104 are attached to the heat exchange portion 104 at the seal connection 142. In various embodiments, the manifold sheets 304, 306 are bonded to the heat exchange portion 104 by heat, radio frequency, impulse or induction sealing, or welding, or chemical or adhesive bonding. Illustrated in FIG. 3B are the two non-porous layers 314, 316 and the air gap 352 separating the two non-porous layers 314, 316 from the edges of the manifold sheets 304, 306 that are not connected to the two non-porous layers 314, 316. The air gap 352 extends only from the sealed connection 142 to the end of the sheets 304, 306 at the central cutout portion. Illustrated in FIG. 3C are the seal connections 142 that attach the manifold sheets 304, 306 to each of the two non-porous layers 314, 316.

The two manifold sheets 304, 306 each have a perimeter that is joined to form an outside seam 210. The outside seam 210 forms a seal that is breached for the inlet 102 and the outlet 106, otherwise, the outside seam 210 seals the fluid 202, 204, 108, 206, 208 within the heat exchange pad 10. In various embodiments, the manifold sheets 304, 306 are bonded together by heat, radio frequency, impulse or induction sealing, or welding, or chemical or adhesive bonding along an outer perimeter 210 to form an outer circumferential seal that prevents fluid leakage from the perimeter portions of the heat exchange pad 10.

In the illustrated embodiment of FIG. 2, the inlet 102 and outlet 106 are formed around an inlet port 112 and an outlet port 116. The ports 112, 116 30 connect to flexible tubing that carries the fluid 202, 208 into and out of the heat exchange pad 10. Those skilled in the art will recognize that the ports 112, 116 can connect to the flexible tubing in any number of ways to ensure a fluid-tight connection without departing from the spirit and scope of the present invention. In another embodiment, the ports 112, 116 are tubes connected to the inlet 102 and outlet 106.

Referring to FIGS. 3A, 3B, and 3C, the manifold sheets 304, 306 are joined to form the manifold 122, 132. The outside seam 210 forms one boundary and the edge 322, 324 of the porous layer 302 forms another boundary. Inside the manifold 122, 132, the manifold sheets 304, 306 are joined to form the alternating flow diverters 124 that separate the outside flow channel 332, the middle flow channel 334, and the inside flow channel 336. The inside flow channel 336 communicates directly to the porous layer 302. As illustrated in FIG. 2, fluid 202 entering the inlet 102 is dispersed between the three flow channels 332, 334, 336 in the inlet manifold 122. The fluid 204 flows in the three flow channels 332, 334, 336 from one end of the manifold 122 to the other. The fluid 204 flowing in the outside flow channel 332 and the middle flow channel 334 moves to the end of the manifold 122 opposite the inlet 102. Along the length of the manifold 122, the fluid 204 flows from the outside flow channel 332 to the middle flow channel 334 and then to the inside flow channel 336, where the fluid 204 enters the porous layer 302. In various embodiments, the flow diverters 124 are separated from 0.25 to 1.0 inches, which encourages flow in tortuous paths across the width of the manifold 122 and the porous layer 302.

The flow diverters 124 in the manifolds 122, 132 provide very little flow restriction between the inlet 102 and the heat exchange portion 104 and between the heat exchange portion 104 and the outlet 106. The major flow restriction is at the junction of the inside flow channel 336 and the first end 322 of the porous layer 302. For the inlet manifold 122, this flow restriction causes the fluid 204 to spread across and through the manifold 122 instead of immediately entering the porous layer 302 in the vicinity of the inlet 102. Since flow resistance in the porous layer 302 is much greater than the flow resistance in the manifolds 122, 132, the fluid 204 fills the inlet manifold 122 first and then travels along a direct path through the porous layer 302. Thus, the fluid 204 enters the porous layer 302 across its full width and the fluid 108 leaves the porous layer 302 across the full width. In this manner, the full effect of the temperature controlled fluid 108 is felt across whole surface area of the heat exchange portion 104.

FIG. 4 illustrates a plan view of another embodiment of the heat exchange device 10-A with an adjacent inlet 102 and outlet 106. The illustrated embodiment of the heat exchange device 10-A shows the inlet manifold 122 and the outlet manifold 132 each wrapping around a corner of the heat exchange portion 104. Although the illustrated embodiment shows the inlet 102 and outlet 106 positioned next to each other, those skilled in the art will recognize that the inlet 102 and outlet 106 can be located at various positions along each respective manifold 122, 132 without departing from the spirit and scope of the present invention.

In the illustrated embodiment, the porous layer 302 has a non-porous skin 314, 316 and the manifold sheets 304, 306 have their central portion cut out with the perimeter of the cutout portion having a seal connection 142 with the porous sheet 302 near the perimeter of the porous sheet 302. The seal connection 142 ensures that the fluid 204, 108, 206 does not escape the confines of the heat exchange device 10-A. Additionally, the manifold sheets 304, 306 have a seal 402 that joins the manifold sheets 304, 306 near the inlet 102 and the outlet 106 and prevents the inlet fluid 204 from passing directly into the porous layer 302 adjacent the inlet 102 and the outlet fluid 206 from passing directly from the porous layer 302 adjacent the outlet 106. That is, the fluid 202 enters the inlet 102, flows within the inlet manifold 122, turns in the direction of fluid 204, and fills the manifold 122. The outside seam 210, the seal connection 142, and seal 402 form a fluid seal that keeps the fluid 204 within the manifold 122. Seal 402 prohibits fluid flow into the porous layer 302 near the inlet 102 and outlet 106, while the seal connection 142 allows fluid flow into layer 302. Thus the fluid 204 is prevented from entering the porous layer 302 at the ends and must enter the porous layer 302 at the side 322 and the fluid 206 can only leave at the opposite side 324 of the porous layer 302.

FIG. 5 illustrates a plan view of another embodiment of the heat exchange device 10-B covering a portion of a person 502. The illustrated embodiment of the heat exchange device 10-B shows the inlet manifold 122 and the outlet manifold 132 located at opposite ends of the heat exchange portion 104. Two inlets 102, 102′ are located at one end of the heat exchange device 10-B and two outlets 106, 106′ are located at the opposite end.

FIG. 6A illustrates a side view of still another embodiment of the heat exchange device 10-C wrapped around the forearm 610 of a human. The heat exchange device 10-C has dimensions suitable for wrapping about the body part of interest, which can be a forearm, a leg, a hand, or any other portion of a body. In various embodiments, the heat exchange device 10-C has a rectangular, trapezoidal, or an irregular outline that is suitable for wrapping around an object. Those skilled in the art will recognize that the dimensions of the encircling pad 10-C can vary without departing from the spirit and scope of the present invention. Further, those skilled in the art will recognize that the location of the inlet 102 and the outlet 106 can vary without departing from the spirit and scope of the present invention. In various embodiments the heat exchange device 10-C is secured to the body portion 610 with a securing device, for example, adhesives, straps, snaps, hook and loop fasteners, or other means for securing pads wrapped around a body portion 610.

Connected to the inlet port 112 is a length of flexible supply tubing 602 and connected to the outlet port 116 is a length of a flexible exhaust tubing 606. The tubing 602, 606 connects the heat exchange device 10-C to a pump unit 612. The pump unit 612, in one embodiment, includes a fluid pump that moves the fluid 202, 108, 208 through the heat exchange device 10-C. The fluid pump develops sufficient pressure at the required flow rates. In another embodiment, the pump unit 612 includes a temperature conditioning device that conditions the fluid in the pump unit 612 such that the fluid discharging from the outlet of the pump unit 612 and entering the supply tubing 602 is at a specified temperature. Although illustrated with the heat exchange device 10-C of FIG. 6A, the pump unit 612 and the connecting tubing 602, 606 are suitable for use with the other illustrated embodiments of the heat exchange device 10.

In another embodiment, a source of temperature controlled pressurized fluid 202 is provided to the supply tubing 602 and the fluid 208 discharged from the heat exchange device 10 is discarded after flowing from the exhaust tubing 606. In this embodiment, the pump unit 612 is not used. In still another embodiment, the pump unit 612 provides temperature controlled fluid 202 to the heat exchange device 10 and the exhaust fluid 208 is not recirculated by the pump unit 612, but is discarded.

FIG. 6B illustrates a cross-section view of the embodiment of heat exchange device 10-C shown in FIG. 6A. The thickness 622 of the heat exchange portion 104 is illustrated in the cross-section view. When the pump unit 612 supplies pressurized fluid to the heat exchange device 10-C, the heat exchange portion 104 expands and the thickness 622 increases. As the heat exchange portion 104 expands, the inside diameter decreases, thereby providing intimate contact between the heat exchange portion 104 and the body portion 610. Such intimate contact significantly improves the thermal contact and increases the heat exchange efficiency of the heat exchange portion 104 in relation to the body portion 610.

In one embodiment, the heat exchange device 10-C is wrapped around the forearm 610 of a human after the pump unit 612 supplies pressurized fluid to the heat exchange device 10-C. In this embodiment, the heat exchange device 10-C applies only as much compression to the body portion 610 as desired by the person applying the heat exchange device 10-C. In another embodiment, the heat exchange device 10-C is wrapped around the forearm 610 of a human before the pump unit 612 supplies pressurized fluid to the heat exchange device 10-C. In this embodiment, the heat exchange device 10-C applies a compressive force to the body portion 610 when the pump unit 612 supplies pressurized fluid to the heat exchange device 10-C.

In still another embodiment, the heat exchange device 10-C is wrapped around the forearm 610 of a human before the pump unit 612, which includes a temperature conditioning device, supplies pressurized and temperature controlled fluid to the heat exchange device 10-C. The pump unit 612 provides pulsating pressurized fluid to the heat exchange device 10-C. In this embodiment, the heat exchange device 10-C applies heat or cold together with a compressive force to the body portion 610 when the pump unit 612 supplies a pulse of pressurized, temperature controlled fluid to the heat exchange device 10-C and that compressive force is removed after the pulse terminates. The heat exchange device 10-C applies an intermittent compressive force to the body portion 610 that is synchronized with the pressure pulses generated by the pump unit 612. When pressure is applied to the heat exchange device 10-C by a pulse from the pump unit 612, the thickness 622 of the heat exchange portion 104 increases, thereby squeezing the body portion 610 and improving thermal contact between the heat change device 10-C and the body portion 610. For the time between the pressure pulses from the pump unit 612, the thickness 622 decreases, thereby releasing the compressive force on the body portion 610 and reducing thermal contact. By controlling the rate of increase of the pressure, the duration of the pressure pulse, the rate of decrease of the pressure, and the duration between pulses, and by controlling the temperature of the fluid simultaneously, the intermittent compression of the body portion 610 can be used to provide therapeutic value to a patient. The intermittent compression results in the gentle application of force against a patient's skin and can prevent static pressure-induced skin sores and/or can massage an extremity for inducing return blood flow through the body portion 610. In one embodiment, the intermittent compression results in a gradual compression, or progressive wave, moving from the inlet manifold 122 to the outlet manifold 132.

In one embodiment, the outer non-porous layer 314 is a pliable material that has limited stretch and the inner non-porous layer 316 is both pliable and stretchable, or elastic. In this embodiment, after the heat exchange device 10-C is wrapped around the body portion 610, fluid 202 is applied to the device 10-C and the porous layer 302 expands in thickness 622. Because the outer non-porous layer 314 is not stretchable and the inner non-porous layer 316 is stretchable, the inside of the device 10-C, which is bounded by the inner non-porous layer 316, moves inward, applying pressure to the wrapped body portion 610 due to the decreasing inside diameter of the wrapped device 10-C.

In one embodiment, the inlet manifold 122 is located at a distal end of the body portion 610, thereby causing the pressure pulse to propagate through the heat exchange portion 104 as a progressive wave. This progressive wave moving toward the proximal end assists in moving the blood toward the heart.

FIG. 7 illustrates a perspective view of another embodiment of the heat exchange device 10-D adapted to fit on a seat 702. The heat exchange portion 104 is positioned against the sitting portion of the seat 702 such that a person sitting in the seat 702 will contact the heat exchange portion 104. When the device 10-D is pressurized with fluid the heat exchange portion 104 expands, and the soft and pliable thickness of the heat exchange portion 104 provides skin protection by conferring support and pressure reduction around bony prominences such as the sacrum. In the illustrated embodiment, the inlet port 112 and the outlet port 116 are located at the bottom near the floor on which the seat 702 is located. This location allows for unobtrusive routing of the tubing 602, 606. Those skilled in the art will recognize that the inlet 102 and the outlet 106 can be located at other positions on the heat exchange device 10-D without departing from the spirit and scope of the present invention.

In the illustrated embodiment, straps, or attachments, 704 are shown for securing the heat exchange device 10-D to the seat 702. In another embodiment, the heat exchange portion 104 is self-supporting because of the expansion of the porous layer 302 due to the application of pressurized fluid 108.

In this embodiment, the heat exchange device 10-D does not require supporting attachment to the seat 702.

FIG. 8 illustrates a front view of still another embodiment of the heat exchange device 10-E adapted to be worn as an article of clothing, such as a vest, by a human 502. Although the illustrated embodiment is of a vest 10-E, those skilled in the art will recognize that the heat exchange device 10-E can assume the shape of any garment of clothing, for example, a shirt, a vest, pants, shorts, and a dress or skirt, without departing from the spirit and scope of the present invention.

In the illustrated embodiment, the manifolds 122, 132 are located at the edges of the vest 10-E with the inlet port 112 and the outlet port 116 located at the bottom of the vest 10-E. Those skilled in the art will recognize that the ports 102, 106 can be located at other positions on the heat exchange device 10-E without departing from the spirit and scope of the present invention.

FIG. 9 illustrates a plan view of another embodiment of a heat exchange device 10-F. An inlet port 112 provides fluid communication with an inlet section 902, through which the fluid 202 flows into a first manifold channel 904 to the far side of the heat exchange device 10-F where the fluid 202 flows into a second manifold channel 906, which abuts the heat exchange portion 104. The second manifold channel 906 distributes the fluid 204 along one side 322 of the heat exchange portion 104. The fluid 108 flowing within the heat exchange portion 104 exits the heat exchange portion 104 along the opposite side 324 of the heat exchange portion 104 into the outlet manifold channel 914. The fluid 206 flowing within the outlet manifold channel 914 flows into the outlet section 912 and the fluid 208 flows out the outlet port 116.

The inlet section 902, the first manifold channel 904, and the second manifold channel 906 are equivalent to the inlet manifold 122 illustrated in FIGS. 1 to 8. Likewise, the outlet manifold channel 914 and the outlet section 912 are equivalent to the outlet manifold 132 illustrated in FIGS. 1 to 8. The various channels 904, 906, 914 and sections 902, 912 include a pattern of flow diverters 124 formed in the channels 904, 906, 914 and sections 902, 912. The flow diverters 124 help to evenly distribute the fluid 202 as it flows through the second manifold channel 906 into the side 322 of the heat exchange portion 104.

The first and second manifold channels 904, 906 form a folded channel such that the second manifold channel 906 is oriented in the opposite direction as the outlet manifold channel 914 aids in the even distribution of fluid 108 flowing through the heat exchange portion 104. The fluid 202 enters the second manifold channel 906 at a second manifold channel entry end 906-A and the fluid flows along the length of the second manifold channel 906 to a second manifold channel distal end 906-B. As the fluid 202 flows along the length of the second manifold channel 906 it also enters the side 322 of the heat exchange portion 104. On the opposite side 324 of the heat exchange portion 104 the fluid 108 flows into the outlet manifold channel 914 along its length. The fluid 206 flows in the outlet manifold channel 914 from the distal end 914-B to the outlet manifold channel exit end 914-A. The position of the outlet manifold channel exit end 914-A at the furthest point from the second manifold channel entry end 906-A ensures that the fluid 108 is evenly distributed across the full width and length of the heat exchange portion 104. The opposing orientation of the second manifold channel 906 relative to the outlet manifold channel 914 is useful for larger configurations of the heat exchange device 10-F in which the heat exchange portion 104 has a length along the second manifold channel 906 that is greater than the width between the second manifold channel 906 and the outlet manifold channel 914.

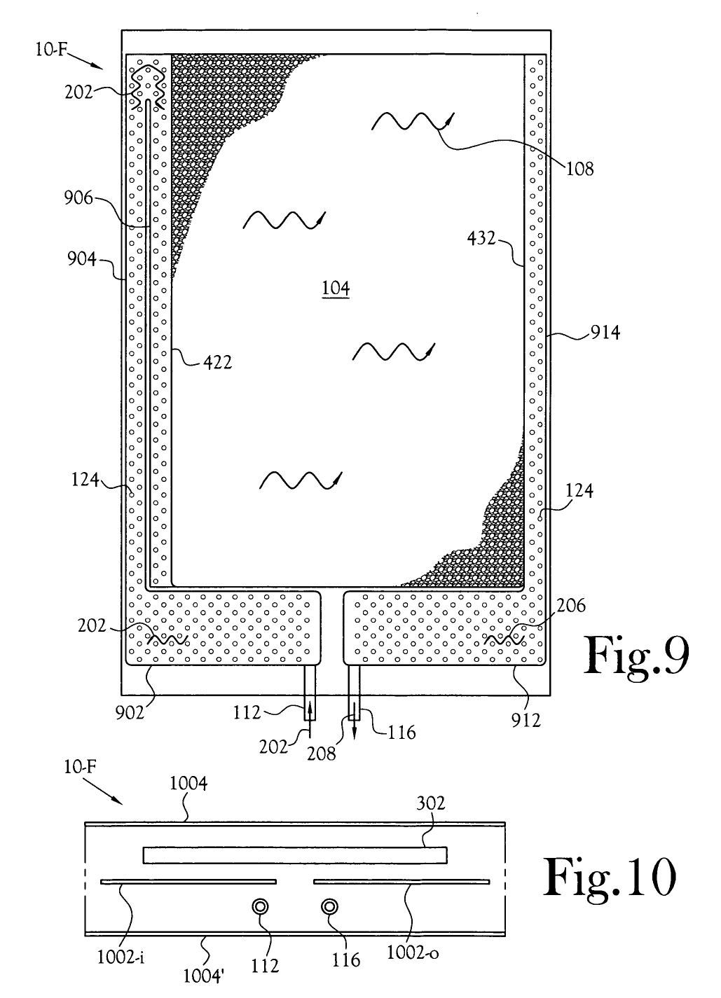

FIG. 10 illustrates an exploded side view of the embodiment of a heat exchange device 10-F illustrated in FIG. 9. The porous layer 302 is sandwiched between two outer non-porous layers 1004 to form the heat exchange portion 104. The two outer layers 1004 extend past the sides 322, 324 of the heat exchange portion 104 to form the various channels 904, 906, 914 and sections 902, 912. In the embodiment illustrated in FIGS. 9 and 10 includes two baffles 1002-i, 1002-o between the top non-porous layer 1004 and the bottom non-porous layer 1004′. The baffles 1002 have a shape that is substantially congruous with the channels 904, 906, 914 and sections 902, 912. In other embodiments, the baffles 1002 are not used and the channels 904, 906, 914, sections 902, 912, and flow diverters 124 are formed by other methods of selectively adhering two non-porous layers 1004 to form fluid tight chambers.

The inlet port 112 and the outlet port 116 are lengths of tubing positioned between the two non-porous layers 1004 such that each one has its inboard end in communication with one of the sections 902, 912. In one embodiment, the inlet and outlet ports 112, 116 terminate at their outboard ends in connectors that allow the ports 112, 116 to be connected to other lengths of tubing 602, 606.

The embodiment illustrated in FIGS. 9 and 10 is fabricated by positioning the porous layer 302, the baffles 1002, and the inlet and outlet ports 112, 116 between the two outer layers 1004. The top non-porous layer 1004 is brought into contact with the bottom non-porous layer 1004′ with the porous layer 302, the baffles 1002, and the inlet and outlet ports 112, 116 sandwiched between the layers 1004. The top non-porous layer 1004 is adhered to the bottom non-porous layer 1004′ and each of the layers 1004 is adhered to the porous layer 302 and the inlet and outlet ports 112, 116. The illustrated construction of the heat exchange device 10-F provides for a device 10-F with an unbroken surface that is not subject to snags or collecting of dirt or other debris.

In operation, when fluid 202 is pumped into the inlet port 112 of the heat exchange device 10-F, the inlet section 902, the first manifold channel 904, and the second manifold channel 906 fill with the fluid 202 and form channels 332, 334, 336 as illustrated in FIG. 3C. The fluid 202 flows into the heat exchange portion 104, thereby causing the porous layer 302 to expand in thickness as it fills with fluid 108. The fluid 108 flows into outlet manifold channel 914 to the outlet section 912 and out the outlet port 116. After the heat exchange device 10-F is completely filled with fluid 202, 108, 206, a thickness equilibrium is reached with the heat exchange portion 104 pressurized and fully expanded. When the flow of the fluid 202 is terminated, the pressure in the heat exchange device 10-F is reduced and the heat exchange portion 104 returns to its normal, unexpanded thickness.

By alternating the pumping of fluid 202 into the heat exchange device 10-F, the heat exchange portion 104 will alternate expanding and reducing in thickness. When the heat exchange device 10-F is used with a patient 502, such alternations of thickness has a beneficial effect on the patient 502. For example, a patient 502 lying on a heat exchange device 10-F for an extended period of time will experience undue pressure on portions of their body, such as the pelvis, where a bony protuberance compresses soft tissue between the protuberance and the device 10-F. By controlling the rate of increase of the pressure, the duration of the pressure pulse, the rate of decrease of the pressure, and the duration between pressure pulses, and by controlling the temperature of the fluid 202 simultaneously, the intermittent changes in the thickness of the heat exchange portion 104 can be used to relieve the compression and/or pressure experienced by the patient 502 and provide therapeutic value.

The heat exchange device 10 includes various functions. The function of distributing flow in the manifolds 122, 132 is implemented, in one embodiment, by the flow diverters 124 formed by connecting the first manifold sheet 304 to the second manifold sheet 306 at selected spots in the manifolds 122, 132. In another embodiment, the function of distributing flow in the manifolds 122, 132 is implemented by the inlet section 902, the first manifold channel 904, and the second manifold channel 906 providing fluid 202 to the heat exchange portion 104 and the outlet manifold channel 914 and the outlet section 912 receiving fluid 108 flowing from the heat exchange portion 104.

The function of containing a porous layer 302 is implemented, in one embodiment, by the two non-porous sheets 314, 316 encasing the porous layer 302. In one embodiment, the two non-porous sheets 314, 316 are fixed to the two surfaces of the porous layer 302, such as with an adhesive or other method. In another embodiment, the porous layer 302 has a non-porous skin that is impermeable to the fluid 108, thereby preventing the fluid 108 from escaping the sides of the porous layer 302. In another embodiment, the function of containing a porous layer 302 is implemented by the two non-porous layers, or sheets, 1004 that encapsulate the porous layer 302.

The function of distributing the fluid 202 to the porous sheet 302 is implemented, in one embodiment, by the inlet manifold 122. In one embodiment, the inlet manifold 122 provides fluid communication between the inlet 102 and the porous sheet 302. In one embodiment, the inlet manifold 122 includes flow diverters 124 that ensure the distribution of the inlet fluid 204 across the end of the porous sheet 302. In another embodiment, the function of distributing the fluid 202 to the porous sheet 302 is implemented by the inlet section 902, the first manifold channel 904, and the second manifold channel 906 providing fluid 202 to the heat exchange portion 104.

The function of receiving the fluid 206 from the porous layer 302 is implemented, in one embodiment, by the outlet manifold 132. In one embodiment, the outlet manifold 132 provides fluid communication between the porous layer 302 and the outlet 106. In one embodiment, the outlet manifold 132 includes flow diverters 124 that ensure the even collection of the fluid 206 across the end of the porous layer 302. In another embodiment, the function of receiving the fluid 206 from the porous layer 302 is implemented by the outlet manifold channel 914 receiving fluid 108 flowing from the heat exchange portion 104.

From the foregoing description, it will be recognized by those skilled in the art that a heat exchange device 10 has been provided. The heat exchange device 10 includes an inlet 102 receiving a temperature controlled fluid 202 that is distributed to and passes through a porous layer 302 before being collected and discharged through an outlet 106. In one embodiment, the porous layer 302 is encapsulated in a pair of non-porous layers 314, 316. In one embodiment, a pair of non-porous layers 304, 306 form a pair of manifolds 122, 132 for distributing the inlet and outlet fluid 202, 208 to and from the porous layer 302. In another embodiment, the porous layer 302 is encapsulated between two non-porous layers 1004 and the non-porous layers 1004 form various channels 904, 906, 914 and sections 902, 912 that allow the incoming fluid 202 to flow within the heat exchange device 10. In one embodiment, the manifolds 122, 132 and/or the channels 906, 914 include flow diverters 124 for distributing the fluid 204, 206 to and from the porous layer 302.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, we claim:

1. A heat exchange apparatus for providing temperature regulation, said apparatus comprising:
a porous layer including an open-cell foam, said porous layer having a first surface, a second surface opposite said first surface, a first end, a second end opposite said first end, and a thickness, said thickness varying in relation to a fluid pressure in said porous layer;
a first non-porous sheet adjacent to said first surface of said porous layer, said first non-porous sheet being pliable, said first non-porous sheet attached to said first surface of said porous layer over a substantial portion of said first surface;
a second non-porous sheet adjacent to said second surface of said porous layer, said second non-porous sheet being pliable, said second non-porous sheet attached to said second surface of said porous layer over a substantial portion of said second surface;
a first manifold in fluid communication with said first end of said porous layer, said first manifold forming a seal with said first non-porous sheet and said second non-porous sheet, said first manifold including a plurality of flow diverters adjacent said first end of said porous layer;
an inlet for receiving a fluid, said inlet in fluid communication with said first manifold, said fluid being a liquid at a selected temperature before being supplied to said inlet;
a second manifold in fluid communication with said second end of said porous layer, said second manifold forming a seal with said first non-porous sheet and said second non-porous sheet; and
an outlet for discharging said fluid, said outlet in fluid communication with said second manifold and receiving said fluid from said second manifold;
whereby said fluid flows from said inlet into said first manifold, into said first end of said porous layer, through said porous layer, out said second end of said porous layer, into said second manifold, and out said outlet.

2. The apparatus of claim 1 wherein each one of said first and second non-porous sheets have an edge extending beyond said first end of said porous layer, said edge of said first non-porous sheet attached to said edge of said second non-porous sheet to form a sealed connection, said first and second non-porous sheets between said sealed connection and said first end of said porous layer defining a portion of said first manifold.

3. The apparatus of claim 1 wherein said first manifold and said second manifold are formed from said first and second non-porous sheets extending beyond said porous layer.

4. The apparatus of claim 1 wherein said first manifold has an inlet end in fluid communication with said first end of said porous layer, said second manifold has an outlet end in fluid communication with said second end of said porous layer, and said outlet end of said second manifold is located at a position furthest away from said inlet end of said first manifold with said porous layer assuming a planar shape.

5. The apparatus of claim 1 further including a pump unit supplying said fluid to said inlet, said pump supply unit receiving said fluid from said outlet.

6. The apparatus of claim 1 further including a pump unit supplying said fluid to said inlet at a pressure that varies periodically, said thickness of said porous layer increasing in relation to increases in said pressure.

7. The apparatus of claim 1 further including a pump unit supplying said fluid to said inlet at a pressure that varies periodically, and said thickness of said porous layer varies in relation to variations in said pressure.

8. The apparatus of claim 1 further including a securing device for securing said apparatus to an object when wrapping said apparatus around said object.

9. The apparatus of claim 1 further including a plurality of attachments for securing said apparatus to a seat.

10. The apparatus of claim 1 wherein said heat exchange apparatus is adapted to be worn as an article of clothing.

11. A heat exchange apparatus for providing temperature regulation, said apparatus comprising:
an inlet manifold for receiving a fluid, said fluid being a liquid, said inlet manifold formed from two layers of a pliable material that is fluid impermeable, said inlet manifold including a plurality of flow diverters;
a heat exchange portion having a first surface, a second surface opposite said first surface, a first end, and a second end opposite said first end, said heat exchange portion being pliable, said first surface and said second surface being impermeable to said fluid, said inlet manifold in fluid communication with said first end of said heat exchange portion, said heat exchange portion having a porous layer between said first surface and said second surface, said porous layer transporting said fluid from said first end to said second end said porous layer including an open-cell foam; and
an outlet manifold for discharging said fluid after said fluid passes through said heat exchange portion, said outlet manifold in fluid communication with said second end of said heat exchange portion; said outlet manifold formed from two layers of a pliable material that is fluid impermeable.

12. The apparatus of claim 11 wherein said first manifold has an inlet end in fluid communication with said first end of said heat exchange portion, said second manifold has an outlet end in fluid communication with said second end of said heat exchange portion, and said outlet end of said second manifold is located at a position furthest away from said inlet end of said first manifold with said heat exchange portion assuming a planar shape.

13. The apparatus of claim 11 wherein one of said two layers of said pliable material for said inlet manifold, said first surface, and one of said two layers of said pliable material for said outlet manifold are defined by a single sheet of material that is impermeable to said fluid.

14. The apparatus of claim 11 wherein said two layers of said pliable material for said inlet manifold are attached to said heat exchange portion at a sealed connection.

15. The apparatus of claim 11 wherein said two layers of said pliable material for said inlet manifold are attached to said heat exchange portion with a first sealed connection, and wherein said two layers of said pliable material for said outlet manifold are attached to said heat exchange portion with a second sealed connection

16. The apparatus of claim 11 wherein each one of said plurality of flow diverters is defined by a connection between a portion of said two layers of said pliable material of said inlet manifold.

17. The apparatus of claim 11 further including a pump unit supplying said fluid to said inlet manifold, said pump supply unit receiving said fluid from said outlet manifold.

18. The apparatus of claim 11 wherein said fluid is conditioned to a selected temperature before being supplied to said inlet manifold.

19. The apparatus of claim 11 wherein said heat exchange portion has a thickness whereby said thickness increases when said fluid enters said heat exchange portion, thereby improving thermal contact and increasing heat transfer between said heat exchange portion and an object adjacent to said heat exchange portion.

20. The apparatus of claim 11 further including a pump unit supplying said fluid to said inlet at a pressure that varies periodically, wherein said heat exchange portion is expandable whereby said heat exchange portion expands in relation to increases in said pressure.

21. The apparatus of claim 11 further including a pump unit supplying said fluid to said inlet at a pressure that varies periodically, wherein said fluid is conditioned to a selected temperature before being supplied to said inlet, and wherein said heat exchange portion is expandable whereby said heat exchange portion expands in relation to increases in said pressure.

22. The apparatus of claim 11 further including a securing device for securing said apparatus to an object when wrapping said apparatus around said object.

23. The apparatus of claim 11 further including a plurality of attachments for securing said heat exchange apparatus to a seat.

24. The apparatus of claim 11 wherein said heat exchange apparatus is adapted to be worn as an article of clothing.

25. A heat exchange apparatus for providing temperature regulation, said apparatus comprising:
a first non-porous sheet impermeable to a fluid, said first non-porous sheet being pliable, said first non-porous sheet having a first perimeter;
a second non-porous sheet impermeable to said fluid, said second non-porous sheet being pliable, said second non-porous sheet having a second perimeter;
a porous sheet for transporting said fluid from a first end to a second end, said porous sheet being pliable, said porous sheet being sandwiched between said first and second non-porous sheets, said first and second perimeters connected to form a seal between said first and second non-porous sheets and thereby encasing said porous sheet;
an inlet manifold formed by said first and second non-porous sheets extending beyond said porous sheet at said first end thereby forming an inlet chamber between said first end and said joined first and second perimeters;
an inlet for introducing said fluid into said inlet chamber, said inlet in fluid communication with said inlet manifold;
an outlet manifold formed by said first and second non-porous sheets extending beyond said porous sheet at said second end thereby forming an outlet chamber between said second end and said joined first and second perimeters; and
an outlet for discharging said fluid from said outlet chamber, said outlet in fluid communication with said outlet manifold.

26. The apparatus of claim 25 wherein said inlet manifold has an inlet end in fluid communication with said first end of said porous sheet, said outlet manifold has an outlet end in fluid communication with said second end of said porous sheet, and said outlet end of said outlet manifold is located at a position furthest away from said inlet end of said inlet manifold with said porous sheet assuming a planar shape.

27. The apparatus of claim 25 wherein said porous sheet has a thickness whereby said thickness increases when said fluid enters said porous sheet, thereby improving thermal contact and increasing heat transfer between said porous sheet and an object adjacent to said porous sheet.

28. The apparatus of claim 25 wherein said porous sheet includes an open-cell foam permeable to said fluid.

29. The apparatus of claim 25 wherein at least one of said inlet manifold and said outlet manifold includes a plurality of flow diverters.

30. The apparatus of claim 25 wherein at least one of said inlet manifold and said outlet manifold includes means for distributing said fluid.

31. The apparatus of claim 25 wherein at least one of said inlet manifold and said outlet manifold includes a plurality of flow diverters, each one of said plurality of flow diverters defined by a connection between a portion of said first and second non-porous sheets.

32. The apparatus of claim 25 further including a pump unit supplying said fluid to said inlet, said pump supply unit receiving said fluid from said outlet.

33. The apparatus of claim 25 wherein said fluid is conditioned to a selected temperature before being supplied to said inlet.

34. A heat exchange apparatus for providing temperature regulation, said apparatus comprising:
a porous sheet of an open cell foam;
a means for containing said porous sheet;
a means for distributing said fluid to said porous sheet; and
a means for receiving said fluid from said porous sheet.

35. A method for using a heat exchange device for providing temperature regulation, said method comprising the steps of:
a) applying a fluid to an inlet manifold of the heat exchange device, said fluid being a liquid, said heat exchange device including said inlet manifold, a heat exchange portion, and an outlet manifold, said inlet manifold in fluid communication with said heat exchange portion, said heat exchange portion in fluid communication with said outlet manifold, said inlet manifold including a plurality of flow diverters, and said heat exchange portion including a porous layer of open-cell foam;
b) maintaining a fluid flow through the heat exchange device for a first selected time;
c) stopping said fluid flow through the heat exchange device for a second selected time; and
d) repeating said steps a), b), and c) for a selected number of repetitions.

36. The method of claim 35 further including the step of selecting a temperature of said fluid before said step a) of applying said fluid.

37. The method of claim 35 wherein said step a) of applying said fluid flow includes the step of increasing said fluid flow at a selected rate.

38. The method of claim 35 wherein said step c) of stopping said fluid flow includes the step of decreasing said fluid flow at a selected rate.

39. The method of claim 35 further including the step of positioning the heat exchange device in relation to a patient to be treated, said step of positioning occurring before said step a) of applying said fluid, and said step of positioning not repeated by said step d).

40. The method of claim 35 wherein said step a) of applying said fluid flow includes the step of applying a pressure until said heat exchange portion has a first thickness, and wherein said step c) of stopping said fluid flow includes the step of decreasing said pressure until said heat exchange portion has a second thickness, said first thickness larger than said second thickness.
